(19) United States
(12) Patent Application Publication
(10) Pub. No.: US 2008/0026460 A1
(43) Pub. Date: Jan. 31, 2008

(54) METHOD FOR CULTURING STEM CELLS

(76) Inventors: Sean P. Palecek, Verona, WI (US); Jeffrey C. Mohr, Madison, WI (US); Juan J. De Pablo, Madison, WI (US)

Correspondence Address: QUARLES & BRADY LLP, 33 E. MAIN ST, SUITE 900, P.O. BOX 2113, MADISON, WI, 53701-2113, US

(21) Appl. No.: 11/765,831

(22) Filed: Jun. 20, 2007

Related U.S. Application Data
(60) Provisional application No. 60/814,975, filed on Jun. 20, 2006.

(51) Int. Cl. C12N 5/02; C12M 1/16; C12N 5/08

(52) U.S. Cl. 435/366; 435/287.9; 435/374; 435/395

(57) ABSTRACT

A three-dimensional microwell system that supports long term embryonic stem cell (ESCs) culture and formation of homogeneous embryoid bodies (EBs) is described. Microwell-cultured ESCs remain viable and undifferentiated for several weeks in culture and maintain undifferentiated replication when passaged to Matrigel®-coated, tissue culture-treated polystyrene dishes. Microwell-cultured ESCs maintain pluripotency, differentiating to each of the three embryonic germ layers. ESC aggregates released from microwells can be passaged for undifferentiated replication or differentiated to monodisperse EBs. The ability to constrain ESC growth in three dimensions advantageously provides for more efficient, reproducible culture of undifferentiated cells, high-throughput screening, and the ability to direct ESC differentiation by generating monodisperse EBs of a desired size and shape.

### CROSS-REFERENCE TO RELATED APPLICATIONS

This application claims the benefit of U.S. Provisional Patent Application No. 60/814/975, filed Jun. 20, 2006, incorporated herein by reference as if set forth in it entirety.

### STATEMENT REGARDING FEDERALLY SPONSORED RESEARCH OR DEVELOPMENT

This invention was made with United States government support awarded by the following agency: NSF DMR-0079983. The United States government has certain rights in this invention.

### BACKGROUND

Embryonic stem cells (ESCs) can proliferate without limit and can differentiate into each of the three embryonic germ layers [1-3]. To facilitate self-renewal, primate (including human) ESCs are typically co-cultured with mouse embryonic fibroblast (MEF) feeder cells, or cultured in MEF-conditioned medium (MEF-CM) on a Matrigel® extracellular matrix.

Cell microenvironment influences embryonic stem cell (ESC) differentiation [4,5]. For example, spontaneous differentiation of ESC cultures occurs along seemingly random pathways during normal cell culture, especially as colony density and size increase [2,6]. Typically, however, ESC differentiation is stimulated either by co-culturing the cells with cells of particular lineages or by chemically or mechanically detaching the cells from their substrate to generate embryoid bodies (EBs) [2] that are cultured to suspension in the absence of MEFs or MEF-CM [7-11]. After several days, EBs in the suspension culture are plated to promote proliferation and further cell differentiation.

Interestingly, EBs in a single culture differentiate to distinct cell lineages. Subtle microenvironment differences in and around individual EBs are thought to affect differentiation of cells in EBs, which then further guide differentiation of other cells by cell-cell contact or by secretion of soluble differentiation factors [6]. One factor that may regulate lineage commitment is EB size [12]. For example, Ng et al. showed efficient generation of hematopoietic cells from “spin EBs” (i.e. EBs generated by centrifugation) having a uniform, yet large size, although the actual number of hESCs aggregating to form these EBs was not known [13]. Smaller spin EBs preferentially differentiated along other lineages. Unfortunately, the art lacks simple methods for producing EBs of consistent and desired size from ESCs.

One way to direct culture of some cell types, including 3T3 fibroblasts [14-17], capillary indothelial cells [18-20], mouse melanoma cells [17] and buffalo rat liver cells [17], is to constrain the cells within a patterned area on a two-dimensional (2-D) monolayer. Micron-scale patterns can be formed in self-assembled monolayers (SAMs) by micro-contact printing alkanethiols that spontaneously assemble via a linkage of a terminal sulfur group to sites on a gold substrate. The SAMs reach equilibrium within one to five hours [19].

Suitable alkanethiols typically contain an eleven to eighteen carbon chain and are capped with a functional group. Depending upon the nature of the functional group, SAMs can attract or repel extracellular matrix (ECM) proteins [16-24]. A common protein-repelling alkanethiol is poly-ethylene glycol (PEG)-terminated alkanethiol containing three to six ethylene glycol groups [16-20]. Tri-ethylene glycol (EG3)-terminated alkanethiols resist protein and extracellular matrix adsorption for approximately eight days, but thereafter begin to break down under typical cell culture conditions [16]. In contrast, several alkanethiols, including methyl -and amine-terminated molecules, attract extracellular matrix proteins [9,21,22,25-27].

Unfortunately, 2-D SAM monolayers are of limited utility for culturing primate ESCs because of the cell's growth nature. Unlike many cells, primate ESCs, including human ESCs, do not grow to confluence as monolayers and are not contact inhibited, but rather build upon themselves to form cell aggregates [28]that spread beyond the constrained areas of the 2-D monolayers. Likewise, initial efforts at 2-D micro-contact printing of Matrigel® on SAM surfaces indicated that this method was not suitable for long-term hESC culture because of substrate instability and because growing colonies could span across unpatterned regions.

With few exceptions, current literature regarding patterned 2-D monolayers focuses primarily on cell attachment and replication to generate confluent monolayers in patterned regions, but does not investigate effects of three-dimensional confined geometries on long term health and stability of cell lines that are not strictly contact dependent. Orner et. al. [36] discussed hESC attachment to laminin-derived peptides deposited in 750 μm squares, but the hESCs were only cultured for two days before cellular analysis. After two days, significant spontaneous generation is unlikely to occur even in suboptimal conditions, and no cell characterization data (e.g., differentiation or viability) was present [32]. Although short-term analysis of selective attachment is useful for screening substrates that permit cell adherence and initial replication, several other requirements exist for use as a robust culture technique with hESCs. That is hESCs must remain viable, undifferentiated, retain ability for undifferentiated proliferation upon passaging and remain pluripotent. Because hESC differentiation does not occur immediately, short-term analysis may not accurately represent hESC response to confinement.

Three-dimensional (3-D) microwells have also been used to study effects of confinement on short-term culture of anchorage-dependent cells. For example, NIH-3T3 fibroblasts were deposited as single cells in microwells 15 μm deep, 75 μm² cross-sectional area [15]. These cells, however, were incubated for only four hours to investigate initial cell attachment and spreading, rather than long-term behavior in microwells. Single epithelial cells were also deposited in 11 μm deep × 10 μm lateral microwells. Unfortunately, cell viability after two days was determined solely by visual cell replication [29]. These studies demonstrated the possibility of cell attachment in microwells, but did not show a marked improvement over prior patterned microwells that also constrained cells for at least two days.

### BRIEF SUMMARY

In a first aspect, the invention is summarized in that a method for culturing ESCs, such as hESCs, include the step of culturing the cells in a microwell defined in an upper surface of coating on a substrate, the microwell supporting growth of viable, substantially undifferentiated ESCs that maintain pluripotency in culture for several weeks. Typically, a plurality of microwell is defined in the coating (e.g., as an array). The coating is sufficiently thick that the microwells defined in the coating have measurable dimensions of length, width and depth that define bottom and side wall surfaces.

The bottom surfaces and portions of the side walls proximal to the bottom surfaces are functionalized, with a cell-attracting material to form a cell-attracting portion of the microwell, while upper portions of the side walls and the upper coating surface between the microwells are functionalized with a cell-repulsing material to form a cell-repulsing portion. Without limitation, the cell-attracting material can be a functionalized extracellular matrix protein material such as Matrigel®. Likewise, and without limitation, the cell-repulsing material can be a protein-resistant SAM. Advantageously, the cells can grow in the microwells, but not outside of the microwells. As such, the size and shape of colonies and aggregates attached to the colonies can be controlled. By establishing consistent cell-attracting and cell-repulsing potions of the microwells, the microwells can be dimension-constrained and the colonies that grow in the dimension-constrained microwells can be substantially uniform from well to well. In the microwells, the ESCs remain substantially and undifferentiated (i.e. greater than about 90% or between about 90% of the cells remain undifferentiated) for at least about three weeks when grown in a non-differentiating medium. The substantially undifferentiated cells retain the ability to self renew and can be plated and passaged like ESCs in conventional culture.

The dimensions of the three-dimensional microwells can be varied as desired or can be uniform from one microwell to another. Microwells of any shape (e.g., round, ovoid and rectangular) are contemplated. The dimensions of the plurality of microwells can be constant (but need not necessarily be equal to one another), such that volume, cell number and shape of colonies cultured in the microwells are substantially consistent among the microwells. Preferably the colonies are monodisperse (i.e. have a narrow size distribution). As used herein, “a narrow size distribution” or “monodisperse” means that the size (i.e. diameter), shape and/or volume of cultured colonies/aggregates within the microwells described herein are within at least 20% of each other, alternatively within at least 15% of each other and alternatively within at least 10% of each other.

In some embodiments, the microwell can have a depth between about 10 μm and about 1000 μm and lateral dimensions (i.e. length and width) between about 50 μm and about 1000 μm on a side, and alternatively can be between about 100 μm and about 500 μm on a side. In certain embodiments, the lateral dimensions of the microwell are substantially identical. Volume per microwell can be consistent, while the dimensions can vary from well to well.

In a second aspect, the invention is summarized in that a method for forming EBs having a narrow size distribution includes the steps of harvesting substantially undifferentiated ESCs from the microwells and culturing the harvested ESCs under differentiating culture conditions until the culture contains differentiated cells. Because the undifferentiated ESCs for use in the EB-forming method can be obtained from dimension-constrained microwells having uniform dimensions, supra, aggregates having a narrow size distribution can be harvested, thereby avoiding a shortcoming of existing EB-forming methods, namely that clumps of ESCs from which EBs are now derived can vary widely in size, volume and cell number. The harvesting step can include harvesting entire colonies or harvesting cell aggregates anchored to colonies in the microwells but unattached to the coating. Colonies can be released by enzymatic treatment. Aggregates can be released from the colonies by gentle shearing without dislodging the colonies, which can be cultured again to yield more cell aggregates. Aggregates released by gentle shearing have a narrow size distribution and yield cultured EBs also having a narrow size distribution. Where the entire culture in inadvertently harvested during the shearing step, the resulting EB is substantially larger than the majority of the EBs and can be discarded or ignored. Advantageously, the cell differentiation profile of EBs can be controlled by controlling the size, shape and volume of the undifferentiated cell cultures that give rise to the EBs.

In a third aspect, the invention is summarized in that a method of cryopreserving ESCs includes freezing substantially undifferentiated, microwell-cultured ESCs as described above. In some embodiments of the third aspect, the microwells are rectangular and have a depth between about 10 microns and about 1000 microns with lateral dimensions between about 50 microns and about 600 microns.

In a fourth aspect, the invention is summarized as cell populations of undifferentiated ESCs colonies or EBs having substantially uniform size and shape. In some embodiments of the fourth aspect, the colonies or cell populations have rectangular lateral dimensions between 50 microns and about 600 microns and depths between about 10 microns and about 1000 microns.

In a fifth aspect, the invention is summarized in that a method for culturing ESCs includes culturing substantially undifferentiated ESCs in a dimension-constrained microwell without subculture for at least about three weeks in a medium that does not promote differentiation, wherein greater than about 90% of the ESCs remain undifferentiated after about three weeks.

### BRIEF DESCRIPTION OF THE DRAWINGS

FIG. 1 depicts manufacture of polymeric substrates containing microwells, in accord with the present invention, and shows resulting substrates and microwells treated as described herein to produce hESC aggregates of defined size, shape and volume.

FIG. 2 depicts a normalized count of diameters of hESCs cultured in microwells having a depth of 50 μm and lateral dimensions ranging from 100 μm to 500 μm compared to hESCs cultured in tissue culture polystyrene (TCPS) dishes.

FIG. 3 are images of hESC aggregates from microwell-cultured hESCs and from Matrigel®-cultured hESCs. hESC aggregates were obtained from hESCs cultured 7 days in 120 μm deep × 100-500 μm lateral microwells or from hESCs cultured 7 days on Matrigel® (TCPS) in CMF+. Scale bars are 300 μm.

FIG. 4 depicts a volume-weighted percentage of diameters of EBs formed after conventional hESC culture (TCPS) and after hESC culture in microwells, as well as the volume-weighted percentage of diameters of microwell-derived hESC aggregates.

### DETAILED DESCRIPTION OF PREFERRED EMBODIMENTS

The present invention relates to the observations that size, shape and volume of undifferentiated ESC colonies often vary and that a colony's differentiation profile varies with these attributes. On the other hand, a controlled 3-D cell microenvironment, achieved by providing chemical and physical constraints on the dimensions the colonies, produces ESCs having a narrow size distribution, thereby allowing one to direct subsequent differentiation by controlling initial ESC size, shape and/or volume. The ability to sustain high density, undifferentiated ESC cultures for weeks without passaging may have valuable applications to general ESC culture techniques. Additionally, the lack of ESC differentiation after several weeks in constrained culture suggests that differentiation is tightly linked to ESC colony size or shape.

Aside from constraining ESC growth, microwell culture facilitated generation of undifferentiated cell aggregates that were easily passaged or differentiated in suspension to form EBs. EB size appears to influence differentiation fate, although the only reported means of controlling EB size involves enzymatically digesting ESC colonies with trypsin to single cells, and then centrifuging the desired number of cells to form a pellet [13]. Interestingly, trypsin inhibits later ESC aggregation and the cell clump formed by centrifugation is morphologically distinct from typical ESC colonies. By using microwell culture, one can define EB size without compromising cell viability or EB structure.

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are described herein.

The invention will be more fully understood upon consideration of the following non-limiting Example. In the Examples, hESC were cultured in microwells, and hESC-derived EBs were obtained. It is specifically contemplated that the methods disclosed are suited for primate ESC generally, as well as other ESCs.

### EXAMPLES

### Example 1

### Microwells for hESCs Culture and Embryoid Body Generation

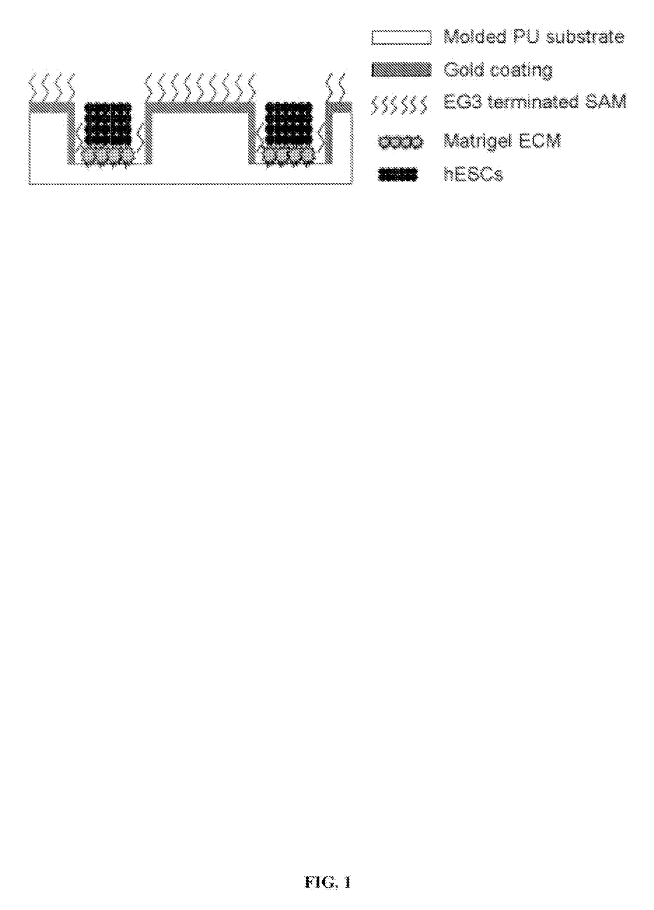

Reference is made to FIG. 1. Microscope slides having formed thereupon a homogeneous distribution of wells of identical size and shape were constructed in three steps using a polydimethylsiloxane (PDMS) stamp to shape a surface of a UV-crosslinkable polyurethane polymer substrate. First, silicon masters, each having desired microwell patterns formed into a surface thereof, were prepared using photolithography and plasma etching techniques similar to those described by Chen et.al. [22], incorporated herein by reference as if set forth in its entirety. The surfaces were passivated by fluorination with (tridecafluoro-1,1,2,2,-tetrahydrooctyl)-1trichlorosilane vapor. Second, a mixture of PDMS elastomer pre-polymer with curing agent (10:1) (Sylgard 184 Silicon Elastomer; Dow Corning, Midland, Mich.) was poured over silicon masters to form PDMS stamps. The mixture was degassed under vacuum and incubated overnight at 70° C. to promote polymerization. The PDMS stamps were then clipped on two sides to glass microscope slides separated by 250 μm spacers. Norland optical adhesive 61 (Norland Products Inc.; Cranbury, N.J.) pre-polymer was fed to one end of the clipped stamps and distributed via capillary action. After cross-linking under UV light for two hours, the stamps and spacers were removed, yielding patterned microwells on the slides with depths of 50 μm-120 μm and lateral dimensions of 50 μm-600 μm. Third, the surfaces of the slides were coated with gold by e-beam evaporation using oblique angles to restrict gold evaporation to the inter-well portions of the surface and to the sides of microwells. Two evaporations were performed, with slides rotated 90° between evaporations. A 80-100 Angstrom titanium layer preceded a 200-500 Angstrom gold layer evaporation. The resulting gold-treated array of microwells was semi-transparent, allowing use of light microscopy during culture. The microwells were washed in 100% ethanol and sterilized under UV light for one hour.

Slides were placed in individual wells of a 6-well culture dish with 2 ml/well of a 2 mM tri-ethylene glycol-terminated (Prochimia; Sopot, Poland) alkanethiol ethanoic SAM solution. Slides were incubated at room temperature for 2 hours and washed in 100% ethanol. All SAM solutions were stored at 4° C. and used within 1 week.

The bottoms of the microwells were then coated with a solution of growth factor-reduced Matrigel® (Beckton-Dickinson; San Jose, Calif.) by re-suspending b 2 mg of Matrigel® in 12-24 ml cold DMEM/F12. About 1 ml of cold Matrigel® solution was then aiquoted to each microwell array and an additional 1 ml of DMEM/F12 was added to each sample to promote cell adhesion to the wells, where gold was not deposited. After 1 hour of incubation at 37° C., the microwells were washed once in PBS and were then transferred to non-tissue culture treated, polystyrene, 6-well plates to prevent cells from attaching to the plate surface around the microwell sides.

hESCs (of lines H1or H9, passage 20-24; WiCell Research Institute; Madison, Wis.) from wells of a 6-well plate at normal passaging confluency were treated with 1 ml/well and 0.05% trypsin, or 1 ml/well 2 mg/ml dispase in DMEM/F12 (Invitrogen; Carlsbad, Calif.) pre-warmed to 37° C. To prevent hESC colonies from dissociating to single cells, plates were monitored under a microscope, and when hESCs at colony edges began to dissociate, trypsin was neutralized with 2 ml/well MEF medium. hESCs were gently washed from the plate and pelleted. The pellet was re-suspended in 0.75 ml/sample MEF-CM supplemented with 4 ng/ml bFGF (CMF+). The hESC were then seeded in aliquots onto 1-2 microwells having 50 μm or 100 μm lateral dimensions, although in subsequent experiments microwells having 600 μm lateral dimension were used, taking care to retain the entire cell solution on top of the slides. Samples were incubated for 30 minutes at 37° C. to allow hESCs to settle into the microwells before adding 1.5 ml/well CMF+. The medium was changed daily thereafter and the cells typically reached confluence with a week.

hESC localized only to the insides of the wells, as visualized by phase contrast microscopy and by Hoechst DNA-binding dye staining. The desired hESC localization was obtained in microwells having lateral dimensions ranging from 50 μm to 600 μm/side. Although bubbles appeared at the interface between the glass slide and polyurethane substrate after several days in culture, microwell integrity remained intact.

Phase contrast and epifluorescence images of differentiation data were obtained on an Olympus IX70 model microscope (Leeds Precision Instruments; Minneapolis, Minn.) using MetaVue 5.0r1 imaging software. Phase contrast, brightfield and epifluorescence images of hESC localization and viability were obtained on a Leica DM ARB microscope (Leica Microsystems; Inc., IL).

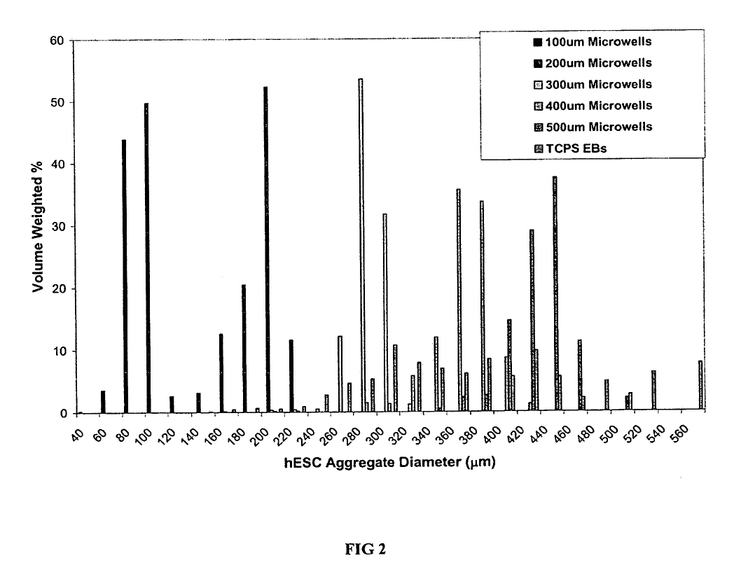

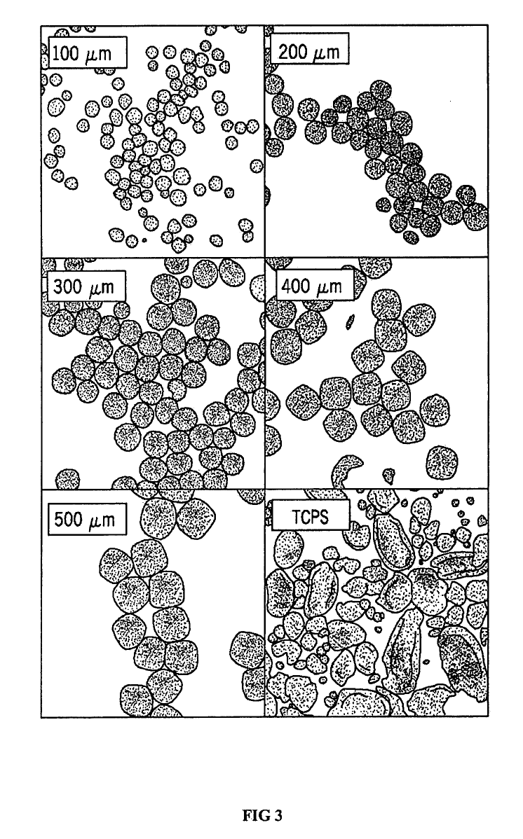

hESCs remained viable and undifferentiated for weeks (i.e. at least 21 days) in microwells. Viability of hESC in microwells was determined by intracellular esterase activity. Live cells having constitutive intracellular esterase activity convert Calcein AM, which readily permeates cell membranes, to the polyanionic dye Calcein, which is retained within the cells and can be detected by fluorescence microscopy. Calcein AM (Molecular Probes; Carlsbad, Calif.) stock was diluted 1:1000 in PBS, aliquoted to confluent hESC microwell cultures on slides, and incubated for 30 minutes at 37° C. The slides were washed 3× and stored in PBS for analysis. As shown in FIG. 2 and FIG. 3, cell dimensions within aggregates corresponded to the size of the microwell in which they were cultured. Larger microwells resulted in cells having larger dimensions.

The microwells (120 μm deep with lateral dimension of 50 μm or 100 μm) contained cells detectable by phase contrast microscopy after 19 days of culture and exhibited Calcein fluorescence. While many live cells were present in the microwell, it was not possible to quantify bell viability using Calcein fluorescence.

To verify the differentiation state of the hESCs, cells were fixed for 15 minutes in 4% paraformaldehyde in PBS with 0.4% Triton X-100. After blocking in 5% milk in PBS+0.4% Triton X-100 for 1 hr at 22° C., primary antibodies were prepared as a 1:200 dilution in PBS+0.4% Triton X-100 and incubated overnight at 4° C. Samples were washed 5 times in PBS before secondary antibodies diluted 1:500 in PBS+0.4% Triton X-100 were added. After 1 hour incubation at 22° C., sampled were washed 3 times in PBS. The primary antibodies used for differentiation analysis were OCT3/4 (Santa Cruz, Biotechnology Inc.; Santa Cruz, Calif.) for undifferentiated hESCs, brachyury (Santa Cruz Biotechnology, Inc.) for mesodermal cells, nestin (Santa Cruz Biotechnology, Inc.) for endodermal cells and α-fetoprotein (Biodesign International; Saco, Me.) for endodermal cells. Alexa fluor 488 or 594 conjugated secondary antibodies (Molecular Probes; Carlsbad, Calif.) were used in all cases. Qualitatively, most cells in microwells expressed OCT4. Several microwells contained multiple layers of cells that were difficult to discern by phase of fluorescence microscopy.

To quantify the differentiation state of hESCs in microwells, cells were harvested after thirteen days and eighteen days of culture. At each microwell depth (50 μm and 120 μm), two lateral dimensions, 50 μm and 100 μm, were analyzed. Cells were removed from the microwells using dispase or trypsin, then were fixed for immunocytochemistry and flow cytometric analysis. hESCs were dissociated from colonies to single cells using a 0.05% trypsin, 0.53 mM EDTA, 2% chicken serum solution. Cells were incubated 15 minutes at 37° C. and trypsin was neutralized using 2 ml/well FACS buffer (PBS without Ca/Mg**, 2% FBS, 0.1% NaN₃). Oct4 expression was quantified by conventional flow cytometry [31 ]. Data were collected on a FACScan flow cytometer (Beckton Dickinson) and analysis was performed on CellQuest (Beckton Dickinson) and WinMD1 software. All living cells were gated according to OCT-4 expression.

To compare differentiation in microwell culture to differentiation under standard culture conditions (i.e., TCPS), expression of OCT4 in hESCs plated on Matrigel® in TCPS dishes and cultured in CMF+ was determined under typical conditions for thirteen and eighteen days, without passaging. hESCs plated on Matrigel® and cultured in CMF+ 6 days prior to fixation were used as positive controls for Oct4 expression. At thirteen days, little difference in Oct4 expression was observed among microwell-cultured cells, TCPS-cultured cells and fresh hESCs. Approximately 90% of cells in each of these culture systems expressed OCT4. After eighteen days, however, clear differences appeared between hESCs cultured in TCPS dishes and those obtained from microwells. Oct4 expression of cells from microwells 50 μm deep with lateral dimensions of 50 μm or 100 μm was 90% and 91%, respectively, compared with 61% for 18-day cells under standard culture conditions (TCPS). Additionally, hESCs cultured on Matrigel®-coated TCPS dishes appeared unhealthy, and colonies were fragmented, with many dead cells floating in medium. Therefore, hESCs constrained to microwell geometries remained undifferentiated for longer periods of time than hESCs cultured in the standard TCPS dish format.

hESCs passaged from microwells to standard cultures maintain undifferentiated replication. Eighteen-day hESC microwell cultures were enzymatically detached using 2 ml/well pre-warmed 10 mg/ml dispase in DMEM/F12 per slide. Plates were incubated for 15 to 25 minutes at 37° C., and cells were washed from microwells by pipeting. The cells on each slide were split to one well of a 6-well TCPS plate coated with Matrigel® and cultured for 5 days in CMF+ prior to immunocytochemical Oct4 expression analysis. For each microwell size and depth measured (50 μm deep with lateral dimensions of 50 μm or 100 μm, and 120 μm deep with lateral dimensions of 100 μm), undifferentiated hESC colonies were passaged to unconstrained TCPS culture in 6-well tissue culture plates with little cell differentiation. After five days, phase contrast microscopy indicated that the unconstrained colonies were much larger than the microwell features, evidencing cell division, and had a morphology typical of colonies continuously cultured in a TCPS dish. hESCs in microwells were fixed in 4% paraformaldehyde for 15 minutes at 22° C. and were then washed 3× in PBS. Hoechst DNA-binding dye (Sigma-Aldrich; St. Louis, Mo.; 10 mg/ml aqueous stock) was diluted 1:1000 in PBS and aliquoted to microwell slides for a 5 incubation at 22° C. Microwell slides were washed 2-3× and store in PBS for epifluorescent analysis. Epifluorescent images of Oct4 expression demonstrated that the vast majority of cells harvested from microwells then cultured in a TCPS dish remained undifferentiated.

Typically, hESC colony differentiation on Matrigel® begins in the colony interior and spreads radially as the colony grows. The differentiation of hESCs cultured in microwells then removed to unconstrained culture was sparse and occurred at the colony edges. The differentiation levels observed are otherwise typical of standard hESC cultures on Matrigel®.

Although hESCs cultured in microwells were typically constrained to the well boundaries, by taking care to minimize shear when exchanging the culture medium, the colonies could be cultured until relatively monodisperse hESC cell aggregates (i.e. a population of aggregates having a narrow size distribution) expanded into the medium above the microwell while remaining attached to the colonies. A typical culture term to form aggregates above microwells 50 μm deep with lateral dimension of 100 μm was 11 days. One could then readily shear the hESC aggregates into the medium by gentle pipetting, leaving behind a confluent hESC base layer in the microwells. If maintained in culture, this base layer replicated to fill the microwell and form a new aggregate.

To assess viability and differentiation state, aggregates were plated on Matrigel®-coated TCPS plates and cultured for 6 days in CMF+. Aggregated attached to the Matrigel ® substrate and began replication within one day. hESC growth rates were consistent with standard hESC cultures and colonies reached passaging confluence within six days. To verify that the hESCs were undifferentiated, colonies were fixed on the sixth day and analyzed for Oct4 expression via immunocytochemistry. Very little differentiation occurred around the colony perimeter, with no visible differentiation in colony interior. This result was similar to results obtained when differentiation of entire hESC colonies enzymatically harvested from microwells was evaluated.

hESCs were cultured on a mouse embryonic fibroblast (MEF) feeder layer in UMF+ (unconditioned hESC medium with 4 ng/ml bFGF; see [31], incorporated herein by reference as if set forth in its entirety) in TCPS dishes for seven days, then cultured in suspension for ten days to induce differentiation and to form conventional “TCPS EBs.” In addition, hESC aggregates were cultured for 14 days in 50 μm deep × 100 μm-600 μm lateral microwells in CMF+ (hESC medium conditioned on mouse embryonic fibroblasts with 4 ng/ml bFGF; see, id.) then cultured in suspension in UMF-(i.e. unconditioned hESC medium without bFGF) in an upright T75 flask for 1 week to induce differentiation to form “microwell-derived EBs.” EBs were plated on 0.1% gelatin-coated 6- or 12-well cell culture plates, cultured in UMF-supplemented with 5% FBS to facilitate attachment. After eight days, the cells were fixed and characterized.

The size distribution of EBs generated from microwell-cultured hESC aggregates and from TCPS-harvested hESC colonies was also examined. Individual EB diameter counts were normalized such that each count was presented as a percentage of total EBs. Microwell-derived EBs exhibited a significantly narrower size distribution that EBs derived from hESCs cocultured with MEFs on TCPS. In general, microwell derived EBs diameters correlated to the size of microwell from which the hESCs were cultured. That is, microwell-derived EBs from hESCs cultured in 100 μm had smaller diameters than microwell-derived EBs from hESCs cultured in 600 μm.

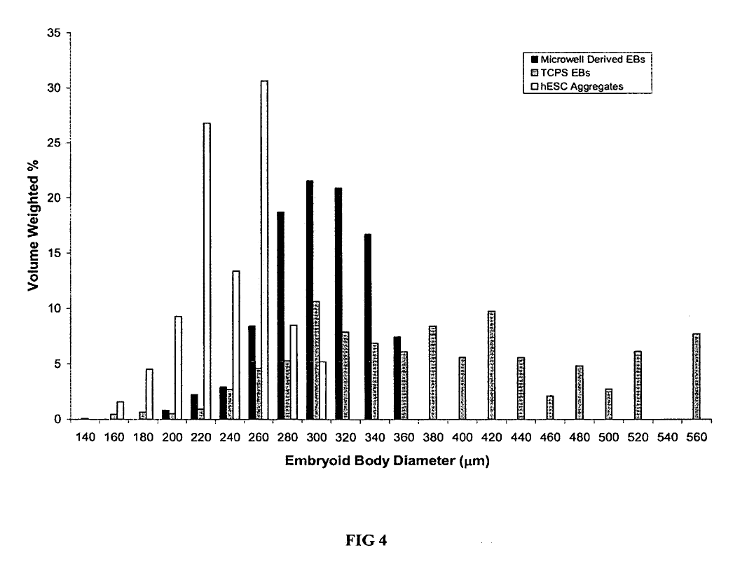

When these data are express in terms of the distribution of volume weighted percentage of EBs of each diameter (FIG. 4), a more pertinent observation is revealed. By accounting for EB volume, which is proportional to the number of cells per EB, it is apparent that typical cell culture methods (which offer little control over hESC colony size) yield a greater percentage of large EBs having substantial volumes and that the distribution of such EB sizes is very wide. In contrast, the narrow size distribution of EBs formed from the microwell-derived hESC aggregates is maintained, as is the diameter of aggregates sheared from such cultures, This is particularly advantageous when attempting to control the parameters affecting differentiation in EBs.

hESCs in microwells, as well as aggregates released into the media, remained viable, undifferentiated and able to be passaged. Pluripotency of hESCs cultured in microwells was assessed using hESC aggregates. EBs were cultured in two ways. First, using standard protocols, hESC aggregates were differentiated in suspension in UMF-containing 5% FBS before plating to gelatin. Second, hESCs aggregates were differentiated by directly plating hESC aggregates harvested from microwells onto gelatin in UMF-containing 5% FBS. Before attaching to the gelatin substrate, hESC aggregates and EBs created from microwells exhibited a relatively monodisperse size distribution. Attachment occurred within two days of plating and EBs were cultured eight days post-attachment. In EBs cultured in suspension for one week and then for eight additional days after attachment, differentiated cultures were fixed and markers characteristic of the three embryonic germ layers using immunocytochemistry targeting the mesodermal marker α-fetoprotein, the ectodermal marker nestin and the endodermal marker brachyury were observed. Similar data were gathered for EBs plated directly to gelatin without culturing in suspension.

### Example 2

### Directed Differentiation of hESCs from Microwell Cultures

hESCs were cultured either in microwells having depths of 50 μm-120 μm and lateral dimensions of 50 μm-500 μm or in TCPS plates as described above. Briefly, samples were incubated for 30 minutes at 37° C. to allow hESCs to settle into the microwells before adding 1.5 ml/well CMF+ to the wells of a 6-well plate. The medium was changed daily thereafter and the cells typically reached confluence within a week. TCPS EBs and microwell-derived EBs were then formed as described above.

Keratinocytes differentiation was achieved by initially forming EBs from microwell-derived hESC aggregates as described above. EBs were grown in suspension for fourteen days in UMF-medium and then attached to gelatin-coated plates in Defined Keratinocyte Serum Free Medium (DSFM) (Invitrogen; Carlsbad, Calif.). Cells were cultured 2-3 weeks prior to analysis by immunocytochemistry and flow cytometry, as described for undifferentiated cells. Keratinocyte marks used include K14 and involucrin.

Cardiomyocyte differentiation began by removing microwell-derived hESC aggregates as described above. EBs were formed by culturing the aggregates in suspension for 1 day in UMF-, followed by culturing for 4 days in cardiac-inducing medium (UMF-, substituted with 20% serum replacer with 20% FBS). EBs were then plated to gelatin-coated TCPS dishes in cardiac-inducing medium for an additional two weeks. Cardio genesis was monitored daily by visual inspection for spontaneously contracting (bearing) regions. The results of which are summarized in Table 1.

TABLE 1

| Microwell Dimension (μm) | Days in Culture | % Beating EBs |
|---|---|---|
| 120 × 100 | 8 | 6.4 |
| 120 × 200 | 8 | 13.9 |
| 120 × 300 | 8 | 14.4 |
| 120 × 400 | 8 | 14.8 |
| 120 × 500 | 8 | 22.7 |
| Control (TCPS) | 8 | 0.5 |

With respect to the EBs differentiated into keratinocytes, the greatest percentage of keratinocytes was observed in cells obtained from microwells having smaller dimensions, such as a depth of 50 μm with lateral dimensions of 100 μm.

With respect to the EBs differentiated into cardiomyocytes, the greatest percentage of cardiomyocytes was observed in cells obtained from microwells having larger dimensions, such as a depth of 120 μm with lateral dimensions of 300-500 μm.

### Example 3

### Microwells for hESCs Culture and Cryopreservation

hESCs were cultured in microwells having dimensions of 50 μm deep with 50 μm-400 μm lateral dimensions as described above. Briefly, samples were incubated for 30 minutes at 37° C. to allow hESCs to settle into the microwells before adding 1.5 ml/well CMF+ to the wells of a 6-well plate. The medium was changed daily thereafter and the cells typically reached confluence within a week. Alternatively, hESCs were cultured in TCPS dishes, as described above.

After 7 days of culture, the hESCs in microwells, suspensions and TCPS plates were place in freezers at −80° C. for up to 4 weeks. Following cryopreservation, cells frozen in suspension were thawed by immersion of the cryovial in a 37° C. waterbath with agitation. Cells were immediately diluted in 10 ml CMF (i.e. hESC medium conditioned on mouse embryonic fibroblasts with bFGF) and centrifuged. hESCs were then diluted in 2 ml CMF and plated to 1 well of a 6-well plated pre-coated with Matrigel® and grown for six days. hESCs frozen in microwells or TCPS plates were thawed by placing the microwells or plates in a 37° C. waterbath with agitation. 3 ml CMF+ (MEF-CM with 4 ng/ml bFGF) were added to each well and then aspirated. 2 ml CMF+ were added to each well and cells were cultured for six days. After six days, cell viability was assessed by intracellular esterase activity as described above. When compared to hESCs cryopreserved on TCPS plates or cryopreserved in suspensions, hESCs cryopreserved in microwells showed significantly greater recover efficiency at all microwell dimensions examined.

The present invention has been described in connection with what are presently considered to be the most practical and preferred embodiments. However, the present invention has been presented by way of illustration and is not intended to be limited to the disclosed embodiments. Accordingly, those skilled in the art will realized that the present invention is intended to encompass all modifications and alternative arrangements within the spirit and scope of the invention as set forth in the appended claims.

### REFERENCES

[1] Reubinoff B, et al., “Embryonic stem cell lines from human blastogysts: somatic differentiation in vitro,” Nat. Biotechnol. 18:399-404 (2000).

[2] Odorico J, et al., Multilineage differentiation from human embryonic stem cell lines,” Stem Cells 19:193-204 (2001).

[3] Thomson J, et al., “Embryonic stem cell lines derived from human blastocysts,” Science 282:1145-1147 (1998).

[4] Spradling A, et al., “Stem cells find their niche,” Nature 414:98-104 (2001).

[5] Streoli C, “Extracellular matrix remodelling and cellular differentiation,” Curr. Opin. Cell Biol. 11:634-40 (1999).

[6] Watt F & Hogan Bm “Out of Eden: stem cells and their niches,” Science 287:1427-1430 (2000).

[7] Schuldiner M, et al., “Effects of eight growth factors on the differentiation of cells derived from human embryonic stem cells,” Proc. Natl. Acad. Sci. USA 97:11307-11312 (2000).

[8] Itskovitz-Eldor J, et al., “Differentiation of human embryonic stem cells into embryoid bodies compromising the three embryonic germ layers,” Mol. Med. 6:88-95 (2000).

[9] Dvash T & Benvenisty N, “Human embryonic stem cells as a model for early human development,” Best Pract. Res. Clin. Obstet. Gynaecol. 18:929-940 (2004).

[10] Tian X, et al., “Cytokine requirements differ for stroma and embryoid body-mediated hematopoiesis from human embryonic stem cells,” Exp. Hematol. 32:1000-1009 (2004).

[11] Wang, L, et al., “Endothelial and hematopoietic cell fate of human embryonic stem cells originates from primitive endothelium with hemangioblastic properties,” Immunity 21:31-41 (2004).

[12] Falconnet D, et al., “Surface engineering approaches to micropattern surfaces for cell-based assays,” Biomaterials 27:3044-3063 (2006).

[13] Ng E, et al., Forced aggregation of defined numbers of human embryonic stem cells into embryoid bodies fosters robust, reproducible hematopoietic differentiation,” Blood 106:1601-1603 (2005).

[14] Lussi J, et al., “Pattern stability under cell culture conditions—a comparative study of patterning methods based on PLL-g-PEG background passivation,” Biomaterials 27:2534-2541 (2006).

[15] Suh K, et al., “A simple soft lithographic route to fabrication of poly(ethylene glycol) microstructures for protein and cell patterning. Biomaterials 25:557-563 (2004).

[16] Luk Y, et al., “Self-assembled monolayers of alkanethiolates presenting mannitol groups are inert to protein adsorption and cell attachment,” Langmuir 16:9604-9608 (2000).

[17] Lehnert D, et al., “Cell behaviour on micropatterned substrata: limits of extracellular matrix geometry for spreading and adhesion,” J. Cell Sci. 117:41-52 (2004).

[18] Mrksich M, et al., “Controlling cell attachment on contoured surfaces with self-assembled monolayers of alkanethiolates on gold,” Proc. Natl. Acad. Sci. USA 93:10775-10778 (1996).

[19] Mrksich M, “Tailored substrates for studies of attached cell culture,” Cell Mol. Life Sci. 54;653-662 (1998).

[20] Chen C, et al., “Geometric control of cell life and death,” Science 276:1425-1428 (2997).

[21] Clare T, et al., “Functional monolayers for improved resistance to protein adsorption: oligo(ethylene glycol)-modified silicon and diamond surfaces,” Langmuir 21:6344-6355 (2005).

[22] Chen C, et al., “Using self-assembled monolayers to pattern ECM proteins and cells on substrates,” Methods Mol. Biol. 139:209-219 (2000).

[23] Chen C, et al., “Micropatterned surfaces for control of cell shape, position, and function,” Biotechnol. Prog. 14:356-363 (1998).

[24] Whitesides G, et al., “Soft lithography in biology and biochemistry,” Annu. Rev. Biomed. Eng. 3:335-373 (2001).

[25] Chen C, et al., “Micropatterned surfaces for control of cell shape, position, and function,” Biotechnol. Prog. 14:356-363 (1998).

[26] Mrksich M, et al., “Using microcontact printing to pattern the attachment of mammalian cells to self-assembled monolayers of alkanethiolates on transparent films of gold and silver,” Exp. Cell Res. 235:305-313 (1997).

[27] Singhi R, et al., “Engineering cell shape and function,” Science, 264:696-698 (1994).

[28] Xu C, et al., “Feeder-free growth of undifferentiated human embryonic stem cells,” Nat. Biotechnol. 19:971-974 (2001).

[29] Dusseiller M, et al., “An inverted microcontact printing method on topographically structure polystyrene chips for arrayed micro-3D culturing of single cells,” Biomaterials 26:5917-5925 (2005).

[30] Chin V. et al., “Microfabricated platform for studying stem cell fates,” Biotechnol. Bioeng. 88:399-415 (2004).

[31] Ji L, et al., “Cryopreservation of adherent human embryonic stem cells,” Biotechnol. Bioeng. 88:299-312 (2004).

[32] Saha S, et al., “Inhibition of human embryonic stem cell differentiation by mechanical strain,” J. Cell Physiol. 206:126-137 (2006).

[33] Noaksson K, et al., “Monitoring differentiation of human embryonic stem cells using realtime PCR,” Stem Cells 23:1460-1467 (2005).

[34] Wei H, et al., “Embryonic stem cells and cardiomyocyte differentiation; phenotypic and molecular analysis,” J. Cell Mol. Med. 9:804-817 (2005).

[35] Sidhu K & Tuch B, “Derivation of three clones from human embryonic stem cell lines by FACS sorting and their characterization,” Stem Cells Dev. 15:61-69 (2006).

[36] Orner B, et al., Arrays for the combinatorial exploration of cell adhesion, “J. Am. Chem. Soc. 126:10808-10809 (2004).

[37] Anderson D, et al., “Nanoliter-scale synthesis of arrayed biomaterials and application to human embryonic stem cells,” Nat. Biotechnol. 22:863-866 (2004).

[38] Mohr J, et al., “3-D microwell culture of human embryonic stem cells,” Biomaterials 27:6032-6042 (2006).

### SEQUENCE LISTING

Not applicable

The invention claimed is:

1. A method for culturing embryonic stem cells to obtain a population of embryoid bodies, the method comprising the steps of:
harvesting substantially undifferentiated embryonic stem cells from a dimension-constrained microwell; and
culturing the harvested embryonic stem cells under differentiating conditions to form the population of embryoid bodies.

2. A method as claimed in claim 1, wherein the population of embryoid bodies have a substantially uniform size and shape.

3. A method as claimed in claim 1, wherein the undifferentiated embryonic stem cells are selected from the group consisting of an embryonic stem cell colony and an aggregate of cells detached from an embryonic stem cell colony.

4. A method as claimed in claim 1, wherein the microwell is rectangular and has a lateral dimension between about 50 microns and about 600 microns.

5. A method as claimed in claim 1, wherein the microwell has a depth between about 10 microns and about 1000 microns.

6. A method as claimed in claim 1, wherein the harvesting step comprises shearing the embryonic stem cells from the microwell.

7. A method as claimed in claim 1, wherein the dimension-constrained microwell has cell-attracting and cell-repulsing portions.

8. An array comprising a substrate having a polymer coating thereupon, the coating defining a microwell on an upon surface thereof, the microwell having side walls and a bottom and having a cell-attractive portion and a cell-repulsing portion.

9. An array as recited in claim 8, wherein the microwells have rectangular lateral dimensions between about 50 microns and about 600 microns and a depth between about 10 microns and about 1000 microns.

10. An array as recited in claim 8, wherein the cell-attractive portion is coated with a cell-attractive material selected from the group consisting of Matrigel, gelatin, laminin, collagen, fibronectin, or a combination of the above.

11. An array as recited in claim 8, wherein the cell-repulsive potion is coated with a cell-repulsing alkanethiol material having a terminal moiety selected from the group consisting of poly-ethylene glycol containing three to six ethylene glycol groups and tri-ethylene glycol.

12. Undifferentiated human embryonic stem cell colonies having a substantially uniform size and shape.

13. The undifferentiated human embryonic stem cell colonies as recited in claim 12, wherein the colonies have rectangular lateral dimensions between about 50 microns and about 600 microns and depth between about 10 microns and about 1000 microns.

14. An embryonic stem cell-derived embryoid body having a substantially uniform size and shape.

15. A method of cryopreserving embryonic stem cells, comprising the step of:
freezing microwell-cultured, embryonic stem cells in microwells.

16. A method as recited in claim 15, wherein the microwells are rectangular and have a depth between about 10 microns and about 1000 microns with lateral dimensions between about 50 microns and about 600 microns.

17. A method for culturing embryonic stem cells, the method comprising the step of:
culturing substantially undifferentiated embryonic stem cells in a dimension-constrained microwell without subculture for at least about three weeks in a medium that does not promote differentiation, wherein greater than about 90% of the embryonic stem cells remain undifferentiated after about three weeks.
